(19) United States
(12) Patent Application Publication
(10) Pub. No.: US 2011/0275095 A1
(43) Pub. Date: Nov. 10, 2011

(54) Microarrays for Allergen-Specific IgE

(75) Inventor: Arthur L. Babson, Chester, NJ (US)

(73) Assignee: SIEMENS HEALTHCARE DIAGNOSTICS INC., Tarrytown, NY (US)

(21) Appl. No.: 13/144,761

(22) PCT Filed: Jan. 14, 2010
(86) PCT No.: PCT/US10/21007

§ 371 Date: Jul. 15, 2011

Related U.S. Application Data
(60) Provisional application No. 61/148,230, filed on Jan. 29, 2009.

(51) Int. Cl. G01N 21/76; G01N 33/566

(52) U.S. Cl. 435/7.9; 436/501

(57) ABSTRACT

Biological samples are assayed for the presence of IgE antibodies specific to unknown allergens in the samples. Known allergens conjugated to biotin are attached as an array of spots on a streptavidin-linked membrane. A sample is incubated with the membrane containing attached known allergens. After washing away excess sample, the membrane is contacted with a labeled anti-IgE, e.g. alkaline phosphatase-labeled anti-IgE, thus attaching anti-IgE to the IgE from the sample, now bound to known allergens. The excess labeled anti-IgE is washed away and the attached IgE remaining on the membrane identified by adding a substrate for the label, thus producing a measurable response.

### BACKGROUND OF THE INVENTION

The invention relates generally to identifying IgE antibodies in biological samples, such as blood. More particularly, the invention is a method and a kit for assaying biological samples to determine which allergen-specific IgEs are present, thus indirectly determining which allergens are causing allergic symptoms in an individual.

There are several hundred substances considered to produce allergic reactions. Many have been isolated and used to identify allergens to which an individual is sensitive. Each allergen is related to a specific IgE antibody produced by the subject's body. Thus it is feasible to identify which of the allergens produces the corresponding IgE found in a sample provided by the subject.

One instrument commercially available is the IMMULITE 1000® from Siemens Healthcare Diagnostics, as described in U.S. Pat. Nos. 5,773,296 and 5,885,529, among others. In that instrument, streptavidin-coated beads are combined with a known allergen that has been biotinylated and a blood sample which may contain IgE antibodies specific for the known allergen. During an incubation, the sample IgE, if present, binds to the biotinylated allergen which, in turn, binds to the streptavidin coated bead. After washing the bead alkaline phosphatase (ALP) conjugated to anti-IgE (ALP/anti-IgE)is added. After incubation to bind the ALP/anti-IgE to the allergen the bead is washed to remove excess unbound ALP/anti-IgE. After adding a chemiluminescent substrate for alkaline phosphatase, the bead is then examined for emitted photons with a photomultiplier tube to determine via the bound ALP/anti-IgE if the known allergen has been attached to IgE in the sample. Each test is for IgE related to a single antigen, and multiple tests are required to determine which allergens an individual has produced IgE antibodies against. Thus, the IgE antibodies produced by the subject have identified their source allergen and appropriate treatment can be undertaken.

U.S. patent publication 2005/0101031A1 discloses a method of detecting immunoglobulin related to certain allergens. An array of allergens are immobilized on a micro-array chip, a sample is incubated with the immobilized allergens to bind immunoglobulins in the sample to specific immobilized allergens. Thereafter, the bound immunoglobulins are detected.

Other methods have been reported from various sources, including Teomed AG, Miragene, Inc., Molecular Staging, Inc. (see U.S. Pat. Nos. 6,531,283 and 6,921,642), DST Diagnostische Systeme & Technologien GmbH, Microtest Matrices Ltd., Hitachi and Phadia. Although the instruments that are available provide for determining single allergen-specific IgEs, there is a need for a simpler and more economical method that provides for the simultaneous determination of multiple allergen-specific IgEs. The present invention provides an improved method of assaying allergens, as will be seen in the description which follows.

### SUMMARY OF THE INVENTION

In broad aspect, the invention is an improved method of detecting which of known allergens create allergic reactions in a subject patient. Known allergens are attached to a membrane and then a sample taken from the patient is applied, so that allergen-specific IgE antibodies in the sample may react with the known allergens. The IgE antibodies are identified by applying labeled anti-IgE, which binds to the IgE antibodies, and then measuring the bound anti-IgE by the response produced when the label is contacted with an appropriate substrate (e.g. chemiluminescence). Which of the known allergens has been bound can be identified from those that are bound to the labeled anti-IgE.

In one embodiment, the method of the invention includes the steps of binding biotinylated known allergens as discrete microspots to a streptavidin-linked membrane and then adding a biological sample suspected to contain allergen-specific IgE. Any allergen-specific IgE in the sample is bound to the related known allergen on the streptavidin-linked membrane. Excess sample is then washed away, after which the membrane is coated with alkaline phosphatase-labeled anti-IgE which binds to any spots containing allergen-specific IgE. After a second wash the membrane is coated with a chemiluminescence substrate for alkaline phosphatase. The light emitted by alkaline phosphatase is measured and correlated with the amount of allergen-specific IgE in the sample.

In another aspect, the invention is a kit for measuring allergen-specific IgE in a sample. A streptavidin-linked membrane having bound to it biotinylated known allergen(s) is used to examine a sample suspected of containing IgE antibodies. Alkaline phosphatase-labeled anti-IgE is used to bind any IgE present in the sample, which has bound to the related known allergen attached to the membrane. A chemiluminescent substrate for alkaline phosphatase is used to produce light that is correlated with the amount of allergen-specific IgE.

### DESCRIPTION OF THE PREFERRED EMBODIMENTS

### Allergen Immunoassays

The purpose of these assays is to identify those allergens that cause a subjects' body to produce characteristic IgE antibodies which, when combined with the allergen, cause the body to produce chemicals, including histamine, and produce allergic symptoms in the subjects' body. Ideally, allergens from various sources can be extracted and/or purified so that they can be used to determine which allergens are causing allergic symptoms in a particular individual. Commercially available assays use such allergens, which have been the subject of numerous patents.

When an array of allergens are contacted with a biological sample e.g. blood, any IgE antibodies produced by the known allergens found in the array will bind to the characteristic known allergen so that one can determine which allergens are producing the IgE antibodies and the associated allergic symptoms.

### Biotinylated Allergens

Allergens may be isolated from their source materials as described in the art. Once isolated, the allergens can be conjugated with linking compounds, such as biotin, by methods known in the art, for example the methods described in U.S. Pat. No. 4,778,751. Thereafter the biotinylated allergens can be contacted with a streptavidin-linked membrane to prepare a medium for determining which allergens in a subject's sample produced related specific IgE antibodies.

In the '751 patent Example 8 provides an illustration of a prior art method in which an avidin coated bead was brought into contact with biotinylated allergen and a biological sample and then, after washing, the bead was reacted with labeled anti-IgE and the amount of allergens determined from the bound IgE antibodies in the sample. This earlier process is related to the commercially available process described above.

### Streptavidin Membranes

An important feature of the invention is the use of membranes having a high density of streptavidin. This means that the membrane has a high capacity for binding biotin. Thus, when the biotin is conjugated to known allergens, the membrane can hold a large number of such allergens. These allergens are then available to bind to IgE antibodies in the subject's blood sample.

One such streptavidin membrane is available from Promega Corporation, designated their SAM²™ membrane. It is reported to have a capacity for biotinylated molecules of 2.5 n mol/cm² . The membrane is believed to be made by proprietary process. The present invention is conveniently described in connection with the Promega streptavidin membrane, but it will be understood that using other membranes is feasible.

### MicroArrays for Allergen-Specific IgE

In a preferred embodiment, a streptavidin membrane will provide a small substrate, say about 2 cm² in area, on which an array of very small spots (about 0.5 mm diameter) of biotinylated known allergens would be deposited by a microarray spotter. Then, blood or other liquid biological sample would be deposited on the membrane containing the known allergens and incubated for about 30 minutes. After incubation, the membrane would be washed with a non-ionic detergent to remove sample residue, leaving allergen-specific IgE from the sample attached to the allergens that were bound to the membrane. ALP-labeled anti-IgE will be contacted with the washed membrane so that the anti-IgE binds to the IgE antibodies from the sample, which now are bound to the known allergens. Excess of the ALP-labeled anti-IgE would be washed away, leaving only the ALP-labeled anti-IgE bound to the IgE that had already been bound to known allergens on the membrane surface. The washed membrane then is contacted with a luminescent substrate for ALP such as Lumagen APS-5 after which the luminescence from ALP is measured using a CCD camera. Alternatively, instead of ALP as a label, horseradish peroxidase may be used, with a suitable chemiluminescent substrate such as Lumigen PS Atto.

In an alternative procedure, the ALP-labled anti-IgE could be combined with the sample before it is applied to the membrane or applied to the membrane with the known allergens before the sample is deposited on the membrane.

Washing away of excess sample or ALP-labeled anti-IgE may be done with various materials, for example a non-ionic detergent containing an inert protein such as bovine serum albumin. The principal consideration in selecting washing material are wash efficiency and prevention of non-specific binding of ALP-labeled anti-IgE.

1. A method of assaying biological samples for allergen-specific IgE comprising:
(a) dispensing on a streptavidin-linked membrane discrete microspots containing biotinylated known allergens, thereby binding said known allergens to said streptavidin-linked membrane;
(b) incubating a biological sample suspected of containing allergen-specific IgE with said streptavidin-linked membrane containing bound known allergens, thereby binding any allergen-specific IgE in said sample to said known allergens on said streptavidin-linked membrane;
(c) washing said sample and said streptavidin-linked membrane incubated in (c) to remove unbound IgE in said sample;
(d) incubating said streptavidin-linked membrane after the washing of step (c) with labeled anti-IgE;
(e) washing said streptavidin-linked membrane after the incubating of (e) to remove unbound labeled anti-IgE;
(f) adding to said washed membrane of (e) a substrate for said label and producing a measurable response;
(g) reading the measurable response of (f) and correlating said response with the amount of allergen-specific IgE in said sample.

2. The method of claim 1 wherein said measurable response is chemiluminescence.

3. The method of claim 2 wherein said label is alkaline phosphatase (ALP) or horse radish peroxidase (HRP).

4. The method of claim 2 wherein said labeled anti-IgE is alkaline phosphatase labeled anti-IgE;

5. The method of claim 3 wherein said substrate for said label is Lumigen APS-5 for ALP or Lumigen PS-Atto for HRP.

6. The method of claim 2 wherein said chemiluminescence is measured with a CCD camera.

7. A kit for assaying biological sample suspected if containing allergen-specific IgE comprising:
(a) a streptavidin-linked membrane with bound microspots of biotinylated known allergens;
(b) labeled anti-IgE;
(c) a substrate for providing a measurable response to said labeled anti-IgE and
(d) a means for measuring the response of said substrate combined with said labeled anti-IgE.

8. A kit of claim 7 wherein said measurable response is chemiluminescence.

9. A kit of claim 8 wherein said chemiluminescence is measured with a CCD camera.

10. A kit of claim 8 wherein said substrate for said label is alkaline phosphatase or horse radish peroxidase.

11. A kit of claim 10 wherein said labeled anti-IgE is alkaline phosphatase-labeled anti-IgE.
